(19) United States
(12) Patent Application Publication
(10) Pub. No.: US 2019/0008367 A1
(43) Pub. Date: Jan. 10, 2019

(54) ENDOSCOPE APPARATUS, ENDOSCOPE SYSTEM AND SURGICAL SYSTEM INCLUDING THE SAME

(71) Applicants: NIKON CORPORATION, Tokyo (JP); MEDICAROID CORPORATION, Kobe-shi (JP)

(72) Inventors: Tetsuro ISHIKAWA, Sakai-shi (JP); Masato HAMATANI, Konosu-shi (JP); Jun NAGATSUKA, Yokohama-shi (JP); Shunji WATANABE, Tokyo (JP); Jiro INOUE, Yokohama-shi (JP); Yasuhiko HASHIMOTO, Kobe-shi (JP); Hirofumi TANAKA, Kobe-shi (JP); Yoshiyuki TAMURA, Kobe-shi (JP); Mitsuichi HIRATSUKA, Kobe-shi (JP)

(73) Assignees: NIKON CORPORATION, Tokyo (JP); MEDICAROID CORPORATION, Kobe-shi (JP)

(21) Appl. No.: 16/129,326

(22) Filed: Sep. 12, 2018

Related U.S. Application Data
(63) Continuation of application No. PCT/JP2016/059672, filed on Mar. 25, 2016.

(51) Int. Cl. A61B 1/00; A61B 1/05; A61B 34/30; A61B 90/50; A61B 34/00; A61B 90/00

(52) U.S. Cl. CPC A61B 1/00149; A61B 1/05; A61B 34/30; A61B 90/50; A61B 1/313; A61B 90/361; A61B 2034/301; A61B 34/70; A61B 34/25

(57) ABSTRACT

This disclosure discloses a surgical system that includes, for example, a patient-side cart (also referred to as a surgical robot) and a console apparatus. The patient-side cart includes an endoscope apparatus that includes three or more endoscope arms. The console apparatus manipulates the patient-side cart. In the endoscope apparatus, for example, feature points of respective images obtained by respective imaging devices of the three or more endoscopes are used to join the respective images together to generate a composite image, and the composite image is displayed on a display screen. The composite image generated from the images in three visual fields to be provided to an operator ensures providing an image in a wide range, and allows the operator to confirm various sites by a visual check during surgery (ensuring an endoscopic surgery while having a large visual field as in a laparotomy) (FIG. 1).

### CROSS REFERENCE TO RELATED APPLICATIONS

This is a Continuation of PCT International Application PCT/JP2016/059672 filed on Mar. 25, 2016. The entire contents of the above document are hereby incorporated by reference into the present application.

### TECHNICAL FIELD

This disclosure relates to an endoscope apparatus and a surgical system that includes the endoscope apparatus.

### BACKGROUND ART

Recently, Minimally Invasive Surgery (MIS) technique has attracted attention. Minimally Invasive Surgery technique is a technique for performing surgery on a patient by using a camera and an elongated surgical instrument introduced into a surgical site inside a body through a small incision site via a trocar sleeve or a cannula. The surgical site often includes a body cavity such as an abdomen of the patient. The body cavity is expanded as necessary using a transparent fluid such as an insufflation gas. Typically, in Minimally Invasive Surgery, an operator such as a doctor uses an end effector of an elongated surgical instrument to operate a handle of the surgical instrument, and operates tissues while observing the surgical site on a video monitor.

A common form of Minimally Invasive Surgery is an endoscopy. A laparoscopy is a kind of the endoscopy to execute a minimally invasive examination and a surgery inside an abdominal cavity. In a typical laparoscopic surgery, a cannula sleeve is passed through a small (generally, equal to or less than ½ inches) incision site to dispose an inlet port for a laparoscopic surgical instrument. A gas is insufflated into an abdomen of a patient to form a space having a certain volume inside the abdominal cavity.

The laparoscopic surgical instrument includes a laparoscope (a kind of an endoscope applied for observing a surgical field inside the abdominal cavity) and operation tools. The operation tools are similar to those used in conventional incision surgery excluding that an operation end or an end effector of each tool is separated from a handle of the tool by a tool shaft.

Relating to such surgical system having the laparoscope, for example, Patent Literature 1 discloses a minimally invasive robotic surgical system where a robot manipulator like a manipulator for moving surgical instruments are used to hold the laparoscopes to align them with a desired surgical site in a patient body.

### CITATION LIST

### Patent Literature

Patent Literature 1: JP 2014-028296 A

### SUMMARY OF INVENTION

In the conventional surgical system as disclosed in Patent Literature 1, a range of images obtained on the surgical site and its peripheral site is limited. For example, it is difficult to obtain an image behind (for example, a part (near a cannula) close to a base of a tubular housing of the laparoscope) the camera disposed on the laparoscope.

(i) This embodiment provides an endoscope apparatus comprising: one or more endoscopes each of that includes an imager on a distal end portion of a tubular housing, and obtains an image in a body of a subject, one or more endoscope arms each of that includes a plurality of joints and a plurality of position detectors corresponding to the respective plural joints, and holds the endoscope, a controller that processes a plurality of images obtained by the one or more endoscopes, wherein the controller obtains position information of the imager by using the plurality of position detectors, and generates a composite image by joining the respective images together by using the position information and feature points of the respective images obtained by the one or more imagers.

(ii) This embodiment also provides an endoscope system, comprising: one or more endoscopes each of that includes an imager on a distal end portion of a tubular housing, and obtains an image in a body of a subject, a robot cart that includes an endoscope apparatus, the endoscope apparatus including one or more endoscope arms each of that includes a plurality of joints and a plurality of position detectors corresponding to the respective plural joints, and holds the endoscope, a console apparatus that transmits an instruction to manipulate the endoscope arm, and a display apparatus that displays an image taken by the endoscope apparatus on a display screen, wherein the endoscope apparatus includes a controller that processes the plurality of images obtained by the one or more of endoscopes, wherein the controller obtains position information of the imager by using the position detectors, and generates a composite image by joining the respective images together by using the position information and feature points of the respective images obtained by the one or more imagers.

(iii) This embodiment provides a surgical system comprising, either one of the above endoscope, wherein the robot cart further includes a surgical arm on which a surgical instrument is mounted, and the console apparatus transmits an instruction to manipulate the surgical arm.

(v) Further features related to this disclosure are clarified from the explanations of this description and the accompanying drawings. The aspects of this disclosure can be achieved and realized by components and combinations of various components, and the following detail description and aspects of accompanying claims. It should be understood that the explanations of this description are merely typical examples and therefore do not limit the claims and application examples of the present invention by any means.

### BRIEF DESCRIPTION OF DRAWINGS

FIG. 1 is a drawing illustrating an exemplary schematic configuration of a surgical system (also referred to as a surgical robot system) 1 according to the embodiment.

FIG. 2 is a drawing illustrating an exemplary internal configuration of a patient-side cart 20 according to a first embodiment.

FIG. 3 is a flowchart describing a composite image generation process in the first embodiment.

FIG. 4 are drawings schematically illustrating visual fields of imaging apparatuses (imagers) to describe a reason why three or more endoscopes are disposed in this disclosure.

FIG. 5 is a drawing illustrating an exemplary configuration of a patient-side cart 20 according to a second embodiment.

FIG. 6 is a drawing illustrating a distal end portion of an endoscope arm 27 on which a transparent sheath 61 having a rounded distal end portion is covered.

FIG. 7 is a drawing describing a visual field ensured in rotating the distal end portion of the endoscope arm 27.

FIG. 8 is a flowchart describing a composite image generation process in the second embodiment.

FIG. 9 is a drawing illustrating an exemplary configuration of a patient-side cart 20 according to a third embodiment.

### DESCRIPTION OF EMBODIMENTS

The following describes embodiments of this disclosure with reference to the accompanying drawings. The accompanying drawings represent functionally identical elements by identical reference numerals in some cases. Although the accompanying drawings illustrate specific embodiments and examples of mounting according to a principle of this disclosure, these drawings are for understanding of this disclosure and never used for limited interpretation of this disclosure.

While the embodiments give the explanation in detail enough for a person skilled in the art to carry out this disclosure, it is necessary to understand that other mountings and forms are possible and that changes in configurations and structures and substitutions of various components can be made without departing from the scope and spirit of the technical idea of this disclosure. Therefore, the following description should not be interpreted to be limited.

Further, as described later, the embodiments of this disclosure may be mounted by software running on a general-purpose computer, by dedicated hardware, or by a combination of software and hardware.

The following describes each process in the embodiments of this disclosure having “each processor (for example, an image processor) as a program” as a subject (operation subject). However, the description may be given with a processor as a subject because the program is executed by the processor (CPU and the like: simply, can be referred to as a controller) to perform specified processes using a memory and a communication port (a communication controller).

### (1) First Embodiment

A first embodiment discloses a surgical system that includes, for example, a patient-side cart (also referred to as a surgical robot) that includes an endoscope apparatus including three or more endoscope arms, and a console apparatus for manipulating the patient-side cart. For example, the endoscope apparatus uses feature points of respective images obtained by respective imaging apparatus (imagers) of the three or more endoscopes to generate a composite image by joining the respective images together, and indicates the composite image on a display screen. The composite image generated from the images in three visual fields and provided to the operator ensures providing the image in a wide range, thus allowing the operator to confirm various sites by a visual check during surgery (ensuring an endoscopic surgery while having a large visual field as in a laparotomy). The three or more endoscopes arranged on appropriate positions ensures obtaining images on approach routes of the endoscopes in real-time when the endoscopes are inserted into a body (into an abdominal cavity or into a thoracic cavity). In this case, it can be confirmed whether the endoscopes squeeze peripheral organs in the body or not.

As a lens of each imaging apparatus (imager) included in the three or more endoscopes, for example, a lens having a viewing angle equal to or more than 90 degrees (for example, a foveal lens) can be used. In the case where the foveal lens is used, the visual field of approximately 180 degrees is ensured, and the center of the visual field is clearly visible while a peripheral region of the visual field is vaguely visible as a visual field of human.

While the approach route of the endoscope can be confirmed in real-time as described above, it is not always possible to insert the three or more endoscopes from optimal positions. Therefore, for example, the images before composition obtained by the respective imaging apparatuses (imagers) since the three or more endoscopes were inserted into a body of a patient (a subject) and the composite images since the three or more endoscopes were inserted into the body of the subject are stored in a memory, for example. Then, in response to an instruction input by the operator, the composite images and the images before composition at a predetermined time point can be indicated on the display screen from the memory. For example, indicating past images of the approach route of the endoscope together with the images during surgery allows the operator to confirm whether a mistake has not been made in the insertion process of the endoscope.

### <Configuration of Surgical System>

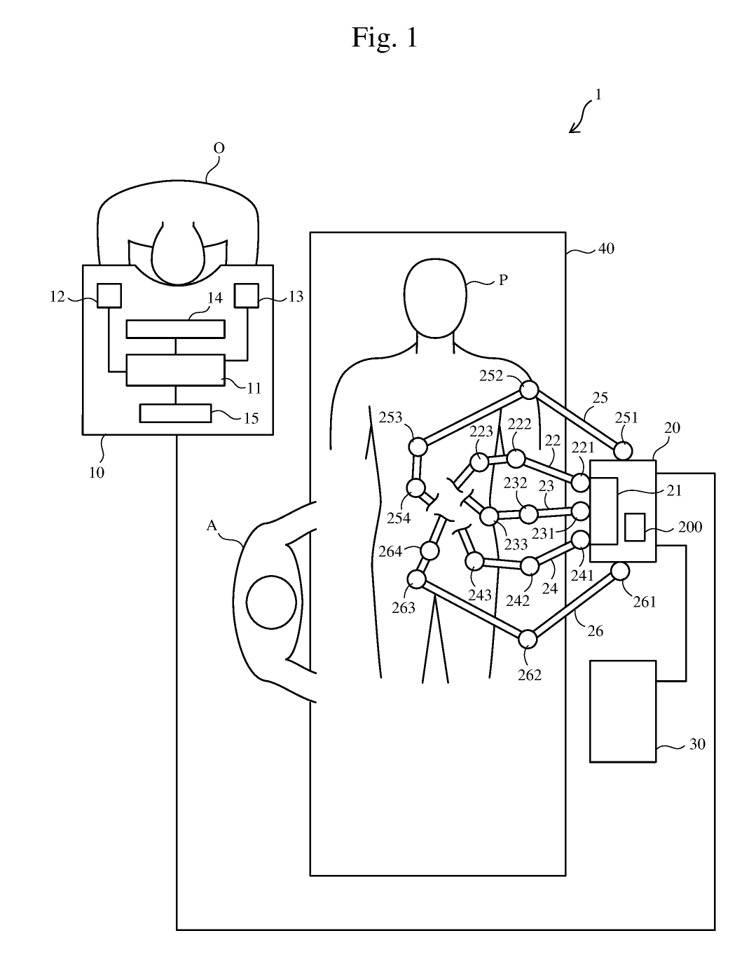

FIG. 1 is a drawing illustrating an exemplary schematic configuration of a surgical system (also referred to as a surgical robot system) 1 according to the embodiment.

The surgical system 1 includes, for example, a console apparatus 10 manipulated by an operator (for example, a surgeon) 0, a patient-side cart (also referred to as a surgical robot cart) 20 for performing a predetermined surgery on a patient (also referred to as a subject) P lying on a surgical table 40 based on instructions from the console apparatus 10, and a display apparatus 30. In FIG. 1, the console apparatus 10, the patient-side cart 20, and the display apparatus 30 are coupled by, for example, wire, while a configuration to couple them by a wireless network (Internet, a wireless LAN, and the like) may be employed.

The console apparatus 10 includes a processor (also referred to as a CPU or a controller) 11 that controls predetermined operations and processes, right and left manipulators 12 and 13 with which the operator O remotely manipulates the endoscope arm and a surgical arm mounted on the patient-side cart 20, a display 14 that indicates images from the endoscope described below, and a communication apparatus 15 that transmits and receives information and data to/from the patient-side cart 20. The console apparatus 10 may include at least one foot pedal (not illustrated). The right manipulator 12 and the left manipulator 13 are, for example, grip input mechanisms that the operator O grips with his/her own hands to manipulate the patient-side cart 20. The operator O can remotely manipulate one or a plurality of surgical arms or endoscope arms mounted on the patient-side cart 20 by manipulating input apparatuses of the right manipulator 12 (the grip input mechanism) and the left manipulator 13 (the grip input mechanism) of the console apparatus 10. This ensures desired operations of the surgical instruments (including end effectors of the surgical instruments) mounted on the surgical arms and the endoscopes mounted on the endoscope arms. Accordingly, the console apparatus 10 functions as a master controller to manipulate the patient-side cart 20 when a desired surgical treatment is executed. In some cases, commands for controlling functions other than the operations of the surgical instruments and the endoscopes mounted on the patient-side cart 20 are provided to the patient-side cart 20 via the console apparatus 10. For example, the foot pedal (not illustrated) can be used to transmit a cautery command for supplying electrosurgical energy to electrosurgical instruments mounted on the surgical arms of the patient-side cart 20 from the console apparatus 10 to the patient-side cart 20. However, the surgical system 1 is not only configured to manipulate the endoscope and the surgical instrument mounted on the patient-side cart 20 via the console apparatus 10, but also may be configured to achieve manipulations of the surgical instruments and the like using, for example, a cart-side manipulator 200 of the patient-side cart 20. For example, in some cases, the endoscope and the surgical instrument of the patient-side cart 20 are manipulated by a surgical assistant A or another operator (for example, a surgeon) who directly manipulates the patient-side cart 20. The input apparatus of the console apparatus 10 may employ an aspect of, for example, a joystick, a motion sensor, a switch, or a thumb/finger control, other than a gripping mechanism or a foot pedal. The above-described “end effector” means an actually operating part (usually, a distal end) of the surgical instrument, and may include, for example, forceps, a grasper, scissors, an anastomosis instrument, an imaging lens, and a needle holder. The end effector for the endoscope (a laparoscope or a thoracoscope) includes, for example, a lens that can be optically coupled to a camera and a lamp via a tool shaft, and light source (in a configuration described below, an illumination light source 212 is included in a main body of an endoscope apparatus 21), in some cases. For executing surgical procedures, the operator O or the surgical assistant A as an operating surgeon passes these operation tools or instruments to a surgical site inside the body via a cannula sleeve, and manipulates them from the outside of the abdomen.

The patient-side cart 20 includes, for example, the endoscope apparatus 21 that includes at least three endoscope arms 22 to 24, at least two surgical arms 25 and 26, and the cart-side manipulator 200. The endoscope arms 22 to 24 and the surgical arms 25 and 26 may be collectively referred to as a patient-side manipulator arm. The at least three endoscope arms 22 to 24 each have a distal end on which an imaging apparatus (imager; for example, a CMOS sensor or a CCD, and can be simply referred to as a camera, the same applies to below) is mounted. The imaging apparatuses (imagers) may be configured to be removable from the respective endoscope arms 22 to 24. On the at least two surgical arms 25 and 26, the surgical instruments are removably mounted corresponding to a surgical technique. The endoscope arms 22, 23, 24 and the surgical arms 25 and 26 include, for example, a plurality of joints 221 to 223, 231 to 233, 241 to 243, 251 to 254, and 261 to 264, respectively. The number of joints to be configured is not limited to the illustrated one, and may be conveniently configured. The joints each include a position detector that detects a rotation direction and rotation angle of each movable component of the arm, and an actuator for driving each movable component (not illustrated), and the position detector and the actuator are mutually associated. The position detector is, for example, an encoder, while a resolver or a potentiometer may be used. Based on information on the rotation direction and the rotation angle of each joint, information on a length between the joints (a length dimension of the movable component, predetermined), a length from the last joint on the arm distal end side to the arm distal end (predetermined), and lengths of the surgical instrument and the imaging apparatus (imager) to be mounted (predetermined), a position of the imaging apparatus (imager) of the endoscope in the body (for example, inside the abdominal cavity and inside the thoracic cavity) of the patient P, and positions of the distal ends of the surgical instruments and predetermined parts in the body of the patient P can be specified. Here, the endoscope is a concept including a laparoscope used for an abdominal surgery and a thoracoscope used for a pulmonary surgery, and can be referred to as, for example, a rigid endoscope.

The display apparatus 30 is installed to, for example, an open place independent of the patient-side cart 20 (not disposed as a display dedicated for the operator O like the display 14 of the console apparatus 10), and indicates the images obtained by the imaging apparatus (imager; for example, the CMOS sensor or the CCD) mounted on the endoscope arms 22 to 24 of the patient-side cart 20 on the display screen. This allows not only the operator O who remotely performs the surgery but also the surgical assistant A and other staff members to confirm a state during surgery. The behavior of the display apparatus 30 may be controlled by, for example, the CPU (also referred to as a processor or a controller) of the patient-side cart 20. In FIG. 1, the display apparatus 30 may be integrally disposed with the patient-side cart 20.

### <Configuration of Patient-Side Cart>

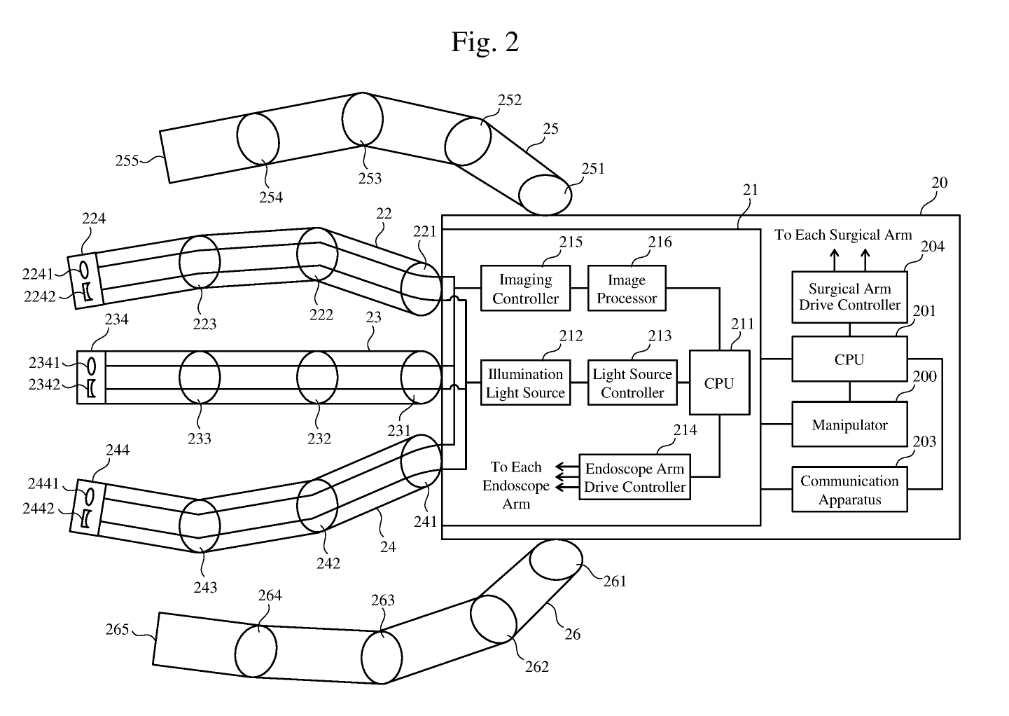

FIG. 2 is a drawing illustrating an exemplary internal configuration of the patient-side cart 20 according to the first embodiment. The patient-side cart 20 is a robot cart including a robot arm, and can be described dividing into the endoscope apparatus 21 that controls the endoscope and the endoscope arm and the other parts that control the surgical arm. A system where a robot cart that includes the endoscope apparatus 21 while not including the surgical arm is remotely manipulated by the console apparatus 10 is an endoscope system, and a system where the robot cart includes the surgical arm as well and the manipulation on the console apparatus 10 manipulates the surgical instrument and the endoscope to perform the surgery is a surgical system.

The endoscope apparatus 21 includes, for example, a CPU (also referred to as a processor or a controller) 211 that controls the entire apparatus, the illumination light source 212 that provides a light source to the imaging apparatus (imager) of the endoscope, a light source controller 213 that controls a behavior of the illumination light source 212 responding to an instruction of the CPU 211, an imaging controller 215 that controls an imaging behavior of the imaging apparatus (imager) of the endoscope, an image processor 216 that processes the images obtained by the imaging apparatus (imager), and an endoscope arm drive controller 214 that drives and controls each of the endoscope arms 22 to 24. On the endoscope apparatus 21 in the first embodiment, for example, at least three endoscope arms 22 to 24 are mounted. The endoscope apparatus 21 may be configured including the endoscope arms 22 to 24. While FIG. 2 is illustrated as a function block diagram of the patient-side cart 20, the light source controller 213, the endoscope arm drive controller 214, the imaging controller 215, the image processor 216, and a surgical arm drive controller 204 may be implemented as programs. In this case, a CPU 201 and the CPU 211 execute various programs to achieve a predetermined processing behavior.

The endoscope arms 22 to 24 include, for example, a plurality of joints 221 to 223, 231 to 233, and 241 to 243, optical adapters 224 to 244, and movable components (no reference numerals) between the respective joints. The plurality of joints 221 to 223, 231 to 233, and 241 to 243 include a plurality of position detectors (not illustrated) (for example, encoders) associated with the respective joints, and the position detectors can each detect a direction and an angle of rotation of each joint. The information on the rotation directions and angles of the respective joints detected by the position detectors is provided to the image processor 216 via the imaging controller 215 or directly without passing through the imaging controller 215. The optical adapters 224 to 244 mounted on tips of the endoscope arms 22 to 24 include imaging apparatuses (imagers) 2241 to 2441, and illuminating optical systems 2242 to 2442 for irradiating with lights from the illumination light source 212. The imaging apparatuses (imagers) 2241 to 2441 are, for example, coupled to the imaging controller 215 via a transmission line (a transmission path: may be wired or wireless). Lenses used for the imaging apparatuses (imagers) 2241 to 2441 are preferred to be lenses that ensure wide visual fields as much as possible (for example, an angle of view (a viewing angle) is 90 degrees or more and 180 degrees or less), and for example, a foveal lens is employed. For example, a fisheye lens may be employed. The illuminating optical systems 2242 to 2442 are, for example, coupled to the illumination light source 212 via optical fibers. The endoscope arms 22 to 24 may further include the joints on distal end portions so as to change imaging directions of the imaging apparatuses (imagers) 2241 to 2441 by behavior of the distal end portions. The endoscope arms 22 to 24 have distal ends (for example, at least portions from the last joints 223 to 243 to the tips, and portions inserted into the body (for example, into the abdominal cavity or into the thoracic cavity) of the patient P) that include tubular housings, and the tubular housings are preferred to have small diameters as much as possible. The smaller the diameter is, the more endoscope arms can be inserted into the body of the patient P, while eliminating the need for suturing after surgery even if the endoscope arms are inserted into the body of the patient P. As the tubular housing, for example, a cylinder-shaped housing is applicable. However, not limited to the cylindrical shape, a tubular housing having another shape (for example, may be an elliptical cylindrical shape or a polygonal cylindrical shape) may be employed.

The light source controller 213 responds to the instruction (for example, given via the CPU 211) given from the operator O and the surgical assistant A to adjust an irradiation light amount (strength) and a color of the illumination light source (for example, an LED, a xenon lamp, a mercury lamp, a halogen lamp, a metal halide lamp) 212. Besides the illumination light source, for example, an LD (a laser diode) may be disposed to cause the LD to function as a laser scalpel for opening the organ and the like of the patient or hemostasis with coherent light. In this case, an output of the LD can be controlled by, for example, the light source controller 213.

The imaging controller 215, for example, outputs a control signal via a signal line coupled to the imaging apparatuses (imagers) 2241 to 2441 to control the imaging apparatuses (imagers) 2241 to 2441. The imaging controller 215, for example, stores image data output from the imaging apparatuses (imagers) 2241 to 2441 in an image memory (not illustrated) with a time stamp.

The image processor 216 obtains the images obtained by the respective imaging apparatuses (imagers) 2241 to 2441 from the image memory (not illustrated). The image processor 216 obtains information (detected by the position detector) on the rotation directions and angles of the plurality of joints 221 to 223, 231 to 233, and 241 to 243, information on the lengths between the respective joints (the movable components), and information on the lengths from the tips of the endoscope arms to the joints closest to the tips, and specifies (calculates) positions of the respective imaging apparatuses (imagers) 2241 to 2441 in the body (for example, in the abdominal cavity or in the thoracic cavity) of the patient P. The information on the lengths between the respective joints (the movable components), and the information on the lengths from the tips of the endoscope arms to the joints closest to the tips are, for example, preliminarily stored in a memory (not illustrated) (a memory area in the image memory may be used). Based on the information on the specified positions of the imaging apparatuses (imagers; for example, information including distances from a reference point, and directions and angles from the reference point) and/or feature points (feature quantities) of the respective images, the image processor 216 joins the images from the three or more imaging apparatuses (imagers) 2241 to 2441 together to generate a composite image. The feature points of the respective images are, for example, can be extracted using Fourier transform or discrete cosine transform, or performing edge filter processing on the images. After extracting the feature points of the respective images, for example, a method for pattern matching can be used to join the respective images together.

The endoscope arm drive controller 214, for example, receives instructions from the CPU 211 to drive motors (not illustrated) disposed in the respective joints 221 to 223, 231 to 233, and 241 to 243 in response to the instructions so as to cause the endoscope arms 22 to 24 to behave as instructed by the manipulators 12 and 13 of the console apparatus 10, or behave as instructed by the cart-side manipulator 200.

The configuration of the patient-side cart 20 includes, other than the endoscope apparatus 21, for example, the CPU (also referred to as a processor or a controller) 201 that controls behavior of the portions other than the endoscope apparatus 21, a communication apparatus 203 for communicating with the console apparatus 10, the cart-side manipulator 200 with which, for example, the surgical assistant A manipulates the patient-side cart 20, the surgical arm drive controller 204 that drives and controls each of the surgical arms 25 and 26, and at least two surgical arms 25 and 26. While the CPU 211 for the endoscope apparatus 21 is separately disposed from the CPU 201 in FIG. 2, one CPU (any one of the CPU 201 and the CPU 211) may control the entire behavior of the patient-side cart 20 including the endoscope apparatus 21.

The surgical arms 25 and 26 include, for example, the plurality of joints 251 to 254 and 261 to 264, the movable components (no reference numerals) between the respective joints, and surgical instrument adapters 255 and 265 on the distal end portions. The plurality of joints 251 to 254 and 261 to 264 include a plurality of position detectors (not illustrated) associated with the respective joints, thus ensuring detecting an angle and a direction of rotation of each joint. The information on the rotation directions and angles of the respective joints detected by the position detectors is provided to the CPU 201.

The cart-side manipulator 200 may employ various aspects other than a gripping mechanism or a foot pedal without being limited, and is configured with, for example, a joystick, a motion sensor, a switch, or a thumb/finger control.

The communication apparatus 203 receives manipulation instructions from the console apparatus 10, and provides the received instructions to the CPU 201 and the CPU 211 of the endoscope apparatus 21. Based on the received instructions, the CPU 201 and the CPU 211 control the behaviors of the endoscope arms 22 to 24 and the surgical arms 25 and 26 of the endoscope apparatus 21.

The surgical arm drive controller 204 receives the commands from the CPU 201 to drive actuators (not illustrated) disposed in the respective joints 251 to 254 and 261 to 265 in response to the commands so as to cause the surgical arms 25 and 26 to execute the commands instructed by the manipulators 12 and 13 of the console apparatus 10, or execute the commands instructed by the cart-side manipulator 200. The actuators are, for example, servo motors.

### <Composite Image Generation Process>

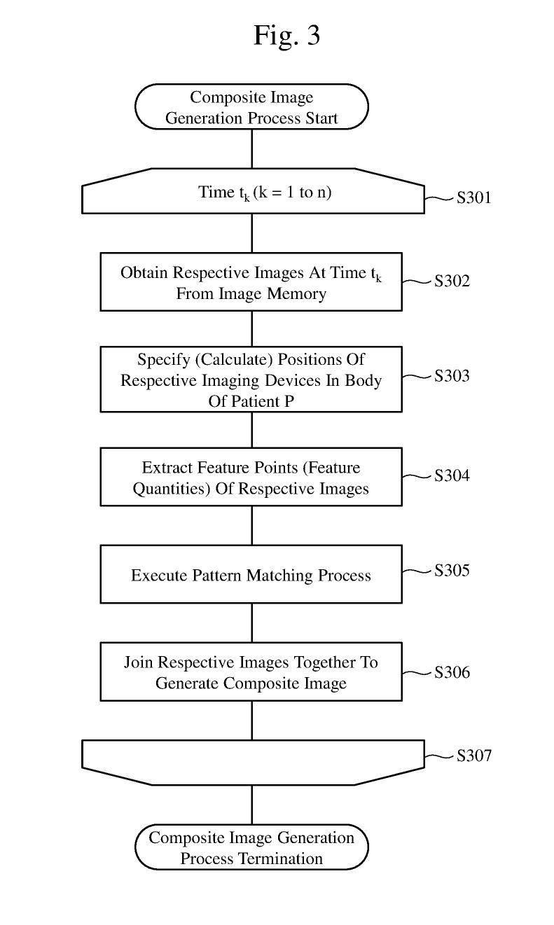

FIG. 3 is a flowchart describing a composite image generation process in the first embodiment. While this composite image generation process is, for example, executed by the image processor 216, when the image processor 216 is implemented by the program as described above, the operation subject is the CPU 211. The following describes the composite image generation process with the image processor 216 as the operation subject, but the description may be understood by replacing the image processor 216 to the CPU 211.

### (i) Step 301 and Step 307

The image processor 216 repeatedly executes processes of Step 302 to Step 306 on each of the images obtained by the imaging apparatuses (imagers) 2241 to 2441 from a time t₁ to a time tₙ. When the composite image generation process is assumed to be executed from a start of a surgery to a termination of the surgery, for example, a surgery start time is defined as t₁ and a surgery termination time is defined as tₙ. When the imaging apparatuses (imagers) 2241 to 2441 obtain images of 30 frames per second for example, a time interval for obtaining each image is 1/30 seconds. The composite image generation process may be executed in units of frame, or in units of field (1 field image per 1/60 seconds).

### (ii) Step 302

The image processor 216 reads images (for example, digital images with three or more frames) obtained by the imaging apparatuses (imagers) 2241 to 2441 at a time tₖ from the image memory (not illustrated). In this Step, the images having three or more frames taken at an identical time are obtained.

(iii) Step 303

The image processor 216 calculates the positions of the imaging apparatuses (imagers) 2241 to 2441 respectively mounted on the distal end portions of the endoscope arms 22 to 24, in the body (for example, in the abdominal cavity or in the thoracic cavity) of the patient P. Specifically, the image processor 216 obtains, for example, the information (detected by the position detector) on the rotation directions and angles of the plurality of joints 221 to 223, 231 to 233, 241 to 243, the information on the predetermined lengths between the respective joints (the movable components), and the information on the predetermined lengths from the tips of the endoscope arms to the joints closest to the tips, from the memory (not illustrated). The image processor 216 calculates the distances and the rotation angles of the respective imaging apparatuses (imagers) from the predetermined reference points based on the information to specify the positions of the respective imaging apparatuses (imagers; for example, the position of the joint 231 is set as the reference point, and a space coordinate is formed with this reference point as the origin, thus the distances and the rotation angles from the reference points of the respective imaging apparatuses (imagers) positioned on the tips of the endoscope arms can be calculated).

### (iv) Step 304

The image processor 216 extracts the feature points of the respective images (The feature point can be referred to as a feature quantity as well. The feature quantity is a feature vector indicating a region around the feature point having great variation of shade with pixel values and differential values). Specifically, the image processor 216, for example, divides the image to be processed into blocks (for example, a block of 8 pixels×8 pixels), and uses Fourier transform, discrete cosine transform, or similar transform to convert the pixel values of the respective blocks into data in a frequency region. With this process, distributions of frequency components in the respective blocks are obtained, thus ensuring extracting the feature points (the feature quantities) of the respective blocks. Besides Fourier transform and discrete cosine transform, an image in which an edge filter is used to emphasize an edge of each block may be configured as the feature point (the feature quantity).

### (v) Step 305

The image processor 216 executes a pattern matching process on respective images. The pattern matching process may be executed on every region of respective images (for example, correlations are calculated on every pixel and block, and a pixel or a block having the highest correlation value is configured as a pattern matching point), while the pattern matching process may be executed with a limited search range. In this case, speeding up and improving the efficiency of the process are ensured. In the case of limiting the search range, based on the information on the positions of the respective imaging apparatuses (imagers) 2241 to 2441 specified in Step 303, the range to search the blocks or the pixels where the feature quantities of the respective images match can be determined (for example, configure the regions of 20% of the peripheries of the respective images as the search ranges). The image processor 216 executes the pattern matching process in the search range, and configures matching positions (positions having the highest correlation) in search regions of the respective images as the pattern matching points.

### (vi) Step 306

The image processor 216 joins the respective images together at the positions recognized as the pattern matching points in Step 305 to generate the composite image. The execution of the pattern matching process detects regions where the respective images mutually overlap. The pixel values of the respective images in the overlapping region are slightly different in some cases (for example, since the imaging directions of the respective imaging apparatuses (imagers) are different, the pixel values obtained by taking images are different in some cases even on an identical object). Then, the images may be joined together after removing the overlapping region from one of the images to be overlapped.

The composite image can be generated with the above-described method. However, the composite image may be generated using a method disclosed in, for example, JP H10-178564 A.

### <Reason for Using Three or More Endoscopes>

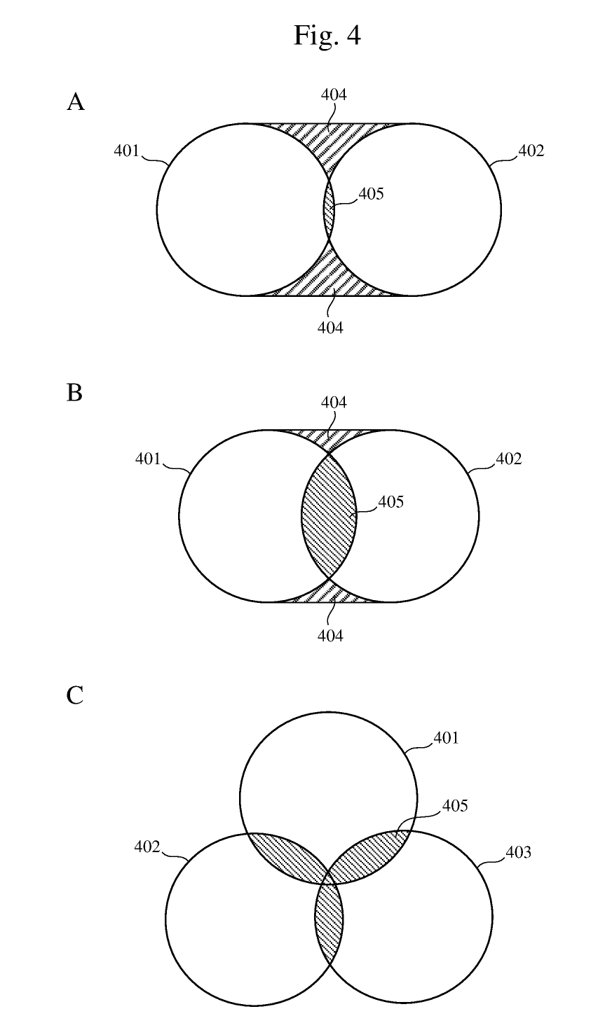

FIG. 4 are drawings schematically illustrating visual fields of the imaging apparatuses (imagers) to describe a reason why three or more endoscopes are disposed in this disclosure. FIG. 4A and FIG. 4B illustrate visual fields when two imaging apparatuses (imagers) are disposed, and FIG. 4C illustrates visual fields when three imaging apparatuses (imagers) are disposed.

For example, when two imaging apparatuses (imagers) are used to obtain images, a visual field 401 and a visual field 402 of the respective imaging apparatuses (imagers) need to avoid overlapping as much as possible (in the case of FIG. 4A, an overlapping region 405 is small). In this case, while an area of the overlapping region 405 can be decreased, blind spots 404 occur between the visual field 401 and the visual field 402. Conversely, as illustrated in FIG. 4B, trying to decrease areas of the blind spots 404 decreases a range covered with the visual field 401 and the visual field 402 to increase the area of the overlapping region 405. In view of this, this cannot obtain a visual field sufficient for obtaining the images to observe a state of the surgery and a state of a periphery of the organ as a target of the surgery in the body (for example, in the abdominal cavity or in the thoracic cavity) of the patient.

Therefore, in this disclosure, three or more imaging apparatuses (imagers) are disposed to use three visual fields, thus attempting to solve the above-described conflicting problem. For example, as illustrated in FIG. 4C, the area of the overlapping region 405 is decreased while eliminating the blind spot 404 formed by overlapping in the case of two visual fields by using the visual field 401, the visual field 402, and a visual field 403 to ensure forming an overall visual field over a wider range.

With the above-described reason, in the first embodiment of this disclosure, the three or more imaging apparatuses (imagers) 2241 to 2441 are used.

### (2) Second Embodiment

A surgical system according to a second embodiment includes a configuration similar to the surgical system according to the first embodiment. However, in the second embodiment, an endoscope apparatus 21 in a patient-side cart 20 has a different configuration.

The second embodiment discloses a surgical system that includes, for example, a patient-side cart (also referred to as a surgical robot) that includes an endoscope apparatus including an endoscope arm configured to have a function to rotate at least a distal end portion of a tubular housing of an endoscope, and a console apparatus for manipulating the patient-side cart. In the endoscope apparatus, for example, an imaging apparatus (imager) has an optical axis having a predetermined angle with a rotation axis of the distal end portion of the tubular housing, images obtained by the imaging apparatus (imager) while rotating the distal end portion of the tubular housing of the endoscope are joined together to generate a composite image, and the composite image is indicated on a display screen. Thus rotating the distal end portion of the endoscope provides the operator with the image of a wide visual field having a position of the endoscope as the center, and the operator can perform a surgery while confirming a state of an affected part and a desired position around the affected part by a visual check (ensuring an endoscopic surgery while having a large visual field as in a laparotomy). In this case, as a lens of the imaging apparatus (imager), for example, a foveal lens can be used as well.

### <Configuration of Patient-Side Cart 20>

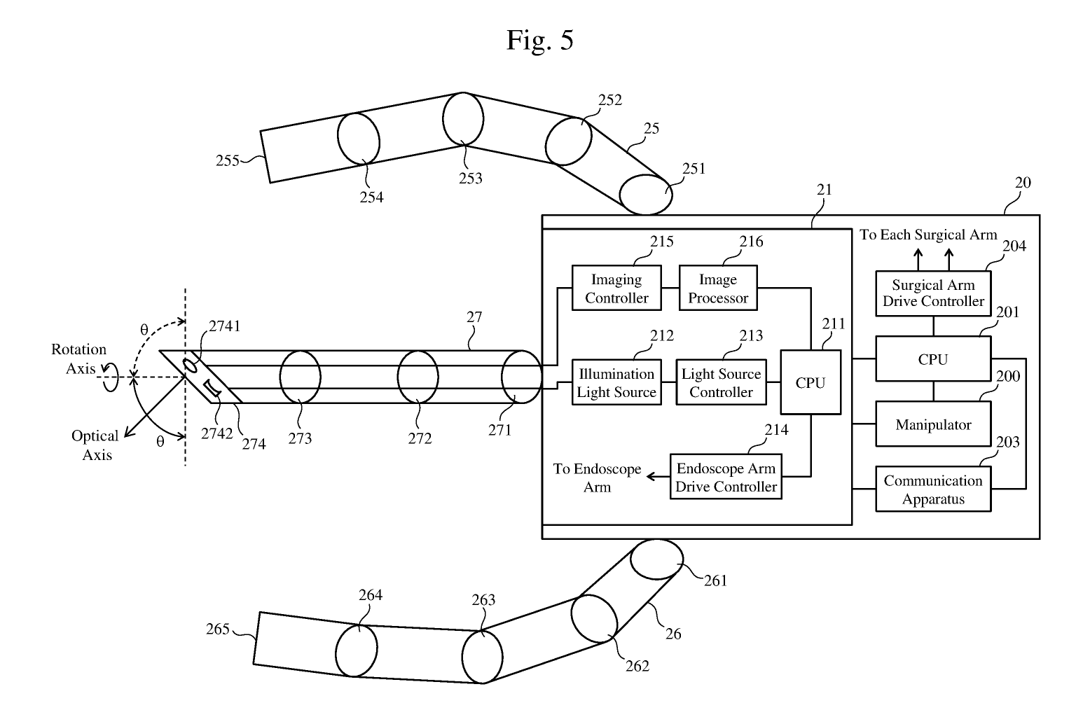

FIG. 5 is a drawing illustrating an exemplary configuration of the patient-side cart 20 according to the second embodiment. A point different from the first embodiment is that one endoscope arm 27 is mounted and the endoscope arm 27 has a distal end portion on which an optical adapter 274 (including an imaging apparatus (imager) 2741 and an illuminating optical system 2742) is configured to be rotatable around a rotation axis. The endoscope arm 27 has an obliquely formed distal end surface, and an optical axis of the imaging apparatus (imager) 2741 has a predetermined angle with the rotation axis. The angle between the optical axis of the imaging apparatus (imager) 2741 and the rotation axis can be configured to be, for example, ½ of an angle of view (a viewing angle) of a lens of the imaging apparatus (imager) 2741. In this case, the distal end surface of the endoscope arm 27 may have an inclined angle of, for example, ½ of the angle of view of the lens of the imaging apparatus (imager) 2741 as well.

In the second embodiment, the imaging apparatus (imager) 2741 obtains images during surgery while the optical adapter 274 on the distal end portion of the endoscope arm 27 is rotated around the rotation axis. The rotation of the optical adapter 274 on the distal end portion of the endoscope arm 27 is controlled by an endoscope arm drive controller 214. Specifically, a joint 273 includes a small (low speed) actuator for rotating the distal end portion separately from an actuator for driving the joint, and the distal end portion of the endoscope arm 27 can be rotated by a rotation drive mechanism (including a rotation drive shaft coupled to the distal end portion and various gears for transmitting rotation drive (for example, a gear such as a bevel gear couples the distal end portion to a shaft of the small motor in some cases)). For example, the endoscope arm drive controller 214 is configured to control a rotation speed and similar factor of the distal end portion. The rotation of the distal end portion of the endoscope arm 27 can be detected by a position detector (for example, an encoder). For example, a position detector disposed on a joint closest to the distal end portion can be used.

The imaging controller 215 stores images obtained by the imaging apparatus (imager) 2741 while rotating in an image memory (not illustrated) with time stamps.

An image processor 216 generates a composite image while performing matching among the images obtained by the imaging controller 215. A composite image generation process will be described later.

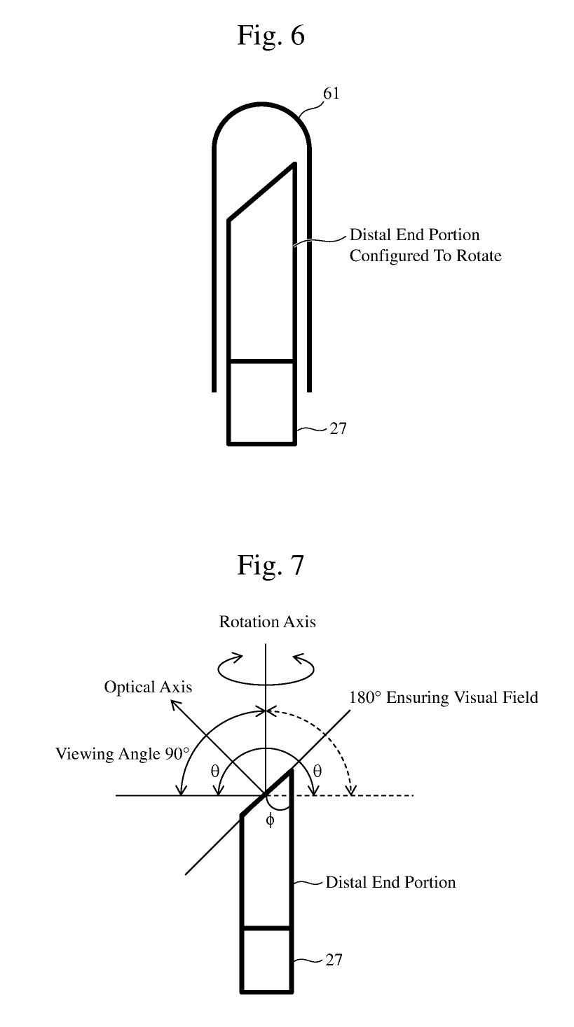

As illustrated in FIG. 6, the distal end portion of the endoscope arm 27 is configured to be covered with a transparent sheath 61 having a rounded distal end portion. This prevents body tissue of the patient P from being damaged during surgery.

### <Visual Field Ensured in Rotating Distal End Portion>

FIG. 7 is a drawing describing a visual field ensured in rotating the distal end portion of the endoscope arm 27.

In FIG. 7, a viewing angle of the lens of the imaging apparatus (imager) is configured to be, for example, 90 degrees. The angle between the optical axis of the lens and the rotation axis is configured to be, for example, 45 degrees. In this configuration, rotating the distal end portion of the endoscope arm 27 around the rotation axis ensures a viewing angle θ of 180 degrees as double of 90 degrees by the rotation, thus ensuring the visual field in a wider range. For example, assuming that the viewing angle of the lens is 120 degrees, the angle between the optical axis and the rotation axis is preferred to be 60 degrees (an inclined angle φ of the distal end surface is 30 degrees). In this case, the viewing angle θ ensured by the rotation is 240 degrees as double of 120 degrees. For example, when a lens having the viewing angle of 180 degrees is disposed on the distal end portion of the endoscope arm 27 such that the angle between the optical axis and the rotation axis is 90 degrees, the viewing angle θ of 360 degrees is theoretically ensured.

### <Composite Image Generation Process>

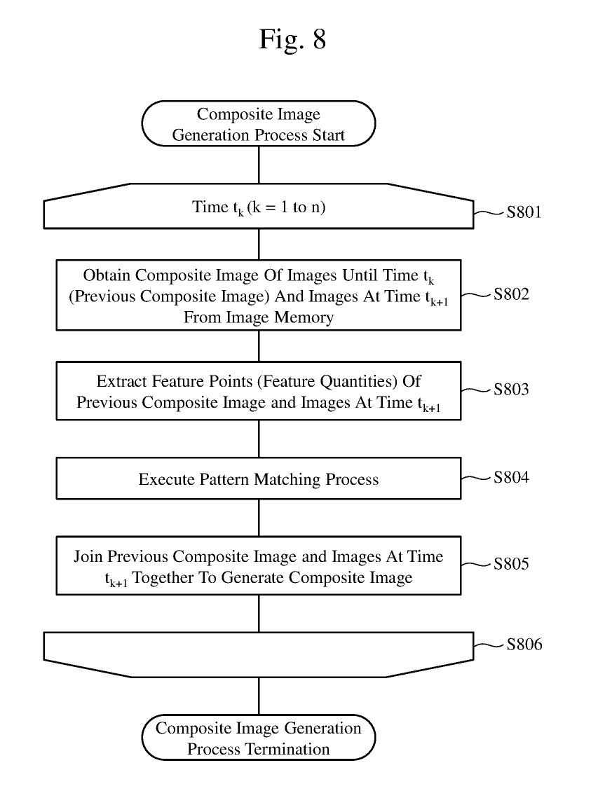

FIG. 8 is a flowchart describing a composite image generation process in the second embodiment. While this composite image generation process is, for example, executed by the image processor 216, when the image processor 216 is implemented by the program as described above, the operation subject is a CPU 211. The following describes the composite image generation process with the image processor 216 as the operation subject, but the description may be understood by replacing the image processor 216 to the CPU 211.

### (i) Step 801 and Step 806

The image processor 216 repeatedly executes processes of Step 802 to Step 805 on each of the images obtained by the imaging apparatus (imager) 2741 from a time t₁ to a time tₙ. When the composite image generation process is assumed to be executed from a start of a surgery to a termination of the surgery, for example, a surgery start time is defined as t₁ and a surgery termination time is defined as tn. When the imaging apparatus (imager) 2741 obtains images of 30 frames per second for example, a time interval for obtaining each image is 1/30 seconds. The composite image generation process may be executed in units of frame, or in units of field (1 field image per 1/60 seconds).

### (ii) Step 802

The image processor 216 reads a composite image (a composite image generated in a previous process: a previous composite image) generated by using the images until a time tₖ and images obtained by the imaging apparatus (imager) 2741 from an image memory (not illustrated). In a case (a case of t₁) where the composite image has not yet been generated at an early stage of the process start, the images between the time t₁ and the time t₂ are read from the image memory instead of the composite image.

(iii) Step 803

The image processor 216 extracts feature points (feature quantities) of the previous composite image and the image at a time tₖ₊₁. Specifically, the image processor 216, for example, divides the image to be processed into blocks (for example, a block of 8 pixels×8 pixels), and uses Fourier transform, discrete cosine transform, or similar transform to convert the pixel values of the respective blocks into data in a frequency region. With this process, distributions of frequency components in the respective blocks are obtained, thus ensuring extracting the feature quantities of the respective blocks. Besides Fourier transform and discrete cosine transform, an image in which an edge filter is used to emphasize an edge of each block may be configured as the feature quantity. When the feature quantity has been already extracted from the previous composite image, for example, it is only necessary to read from the image memory (not illustrated).

### (iv) Step 804

The image processor 216 executes a pattern matching process on the previous composite image and the image at the time tₖ₊₁. In the second embodiment, the rotating imaging apparatus (imager) 2741 ensures the execution of the pattern matching equivalent to the pattern matching on a moving image. Specifically, the image processor 216, for example, preliminarily executes the pattern matching process in a search range (the rotation speed can be used to calculate a moving distance of the image (a shifted range of the image) in one frame period (for example, 1/60 seconds), and the search range can be configured with the range and a margin considering an error), and configures matching positions (positions having the highest correlation) in search regions of the respective images as the pattern matching points.

### (v) Step 805

The image processor 216 joins the previous composite image and the image at the time tₖ₊₁ together at the position recognized as the pattern matching point in Step 804 to generate a new composite image. One rotation of the distal end portion of the endoscope arm 27 generates a composite image of the viewing angle θ. The images obtained by the second rotation or later are sequentially superimposed on an appropriate position (a position where the matching is confirmed) of the already generated composite image.

The composite image can be generated with the above-described method. However, the composite image may be generated using a method disclosed in, for example, JP H10-178564 A.

### <Low Resolution Mode and High Resolution Mode>

In this embodiment, for example, the images obtained while rotating the distal end portion of the endoscope arm 27 are provided in low resolution (low image quality: in other words, a first resolution or a first image quality), and therefore, the composite image obtained by composing those images is also indicated on the display screen of the display apparatus 30 with low resolution. On the other hand, the images obtained while stopping the rotation of the distal end portion are indicated on the display screen of the display apparatus 30 with high resolution (high image quality: in other words, a second resolution or a second image quality (higher resolution compared with the first resolution)).

For example, when the operator O finds an anxious part and the like on the composite image and wants to intensively observe the part, the operator O can manipulate the console apparatus 10 to instruct to stop the rotation of the distal end portion, and instruct the part desired to be further intensively observed to confirm the part on the high resolution image.

The endoscope apparatus and the surgical system according to the embodiment can decrease the number of the endoscope inserted into the body of the patient P compared with the first embodiment, thus making a surgery more minimally invasive.

### (3) Third Embodiment

A surgical system according to a third embodiment includes a configuration similar to the surgical system according to the first embodiment. However, in the third embodiment, an endoscope apparatus 21 in a patient-side cart 20 has a different configuration. In the second embodiment, the endoscope apparatus 21 includes only one endoscope arm 27 having a distal end portion configured to rotate, while the third embodiment is different in a point that three or more endoscope arms having distal end portions configured to rotate are disposed.

The third embodiment discloses a surgical system that includes, for example, a patient-side cart (also referred to as a surgical robot) and a console apparatus for manipulating the patient-side cart. The patient-side cart includes an endoscope apparatus having three or more endoscopes and three or more endoscope arms, and the three or more endoscope arms have a function to rotate at least a distal end portion of a tubular housing of each endoscope. In the endoscope apparatus, for example, imaging apparatus (imager)s each have an optical axis having a predetermined angle with a rotation axis of the distal end portion of each tubular housing, three or more images obtained by the imaging apparatuses (imagers) while rotating the distal end portions of the tubular housings of the respective endoscopes are joined together to generate a composite image, and the composite image is indicated on a display screen. Thus rotating the distal end portions of the three or more endoscopes provides the operator with the image in the visual field of further wide range (for example, 360 degrees), and the operator can perform a surgery while confirming a state of an affected part and a desired position around the affected part in a wide range by a visual check (ensuring an endoscopic surgery while having a large visual field as in a laparotomy). In this case, as a lens of the imaging apparatus (imager), for example, a foveal lens can be used as well.

For example, when the operator instructs it to stop the rotation of the distal end portion of each endoscope arm, the images in the states where the rotations are stopped are indicated on the display screen. In this case, for example, the images obtained and indicated in the stopped state can be provided in high image qualities compared with the images obtained while rotating the distal end portions. Accordingly, the operator can confirm the desired portion in more detail by a visual check. In this case, for example, the operator can give an instruction of information on a position of a target in the body of which the image is to be obtained in the state where the rotation is stopped. At this time, for example, the image obtained by only the imaging apparatus (imager) with which the image of the target position is obtainable is indicated on the display screen.

### <Configuration of Patient-Side Cart>

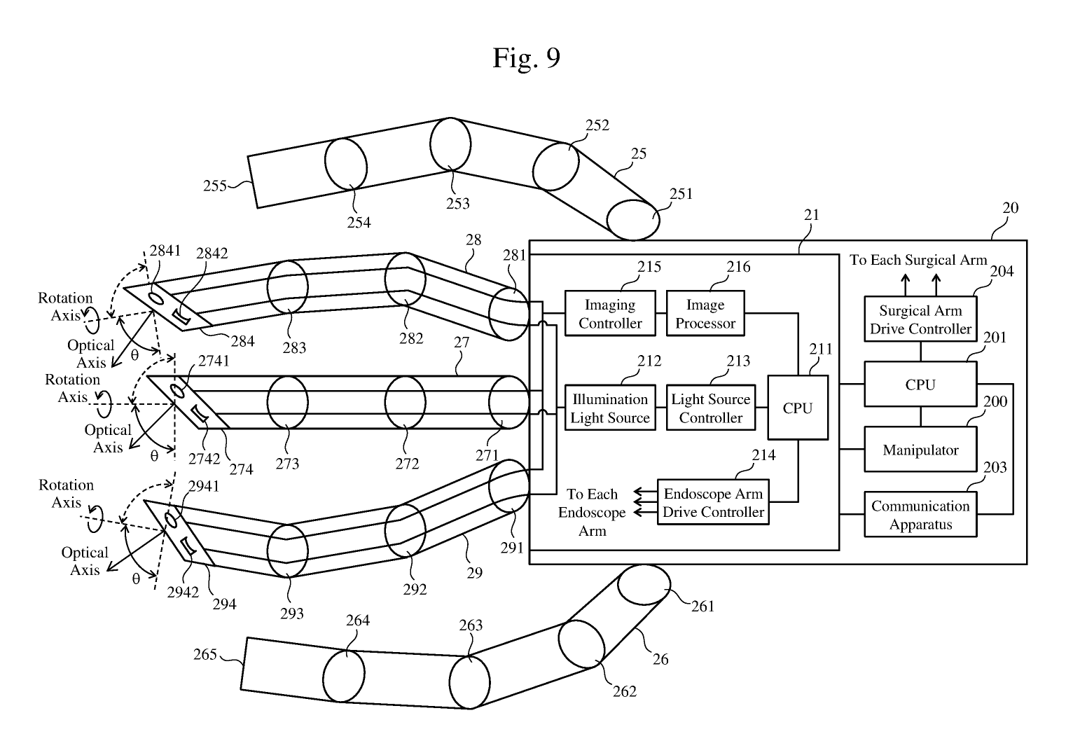

FIG. 9 is a drawing illustrating an exemplary configuration of the patient-side cart 20 according to the third embodiment. A point different from the first embodiment is that three or more endoscope arms 27 to 29 have distal end portions configured to rotate similarly to the second embodiment. A point different from the second embodiment is that the three or more endoscope arms 27 to 29 having the distal end portions configured to rotate are disposed.

Similarly to the second embodiment, the endoscope arms 27 to 29 have obliquely formed distal end surfaces, and optical axes of imaging apparatuses (imagers) 2741 to 2941 each have a predetermined angle with each of the rotation axes of the distal end portions. The angles between the optical axes of the imaging apparatuses (imagers) 2741 to 2941 and the rotation axes can be each configured to be, for example, ½ of an angle of view (a viewing angle) of each lens of the imaging apparatuses (imagers) 2741 to 2941. In this case, the distal end surfaces of the endoscope arms 27 to 29 may have an inclined angle of, for example, (90−the viewing angle of the lens of the imaging apparatus (imager) 2741×½) degrees.

In the third embodiment, for example, the imaging apparatuses (imagers) 2741 to 2941 obtain images during surgery while optical adapters 274 to 294 on the distal end portions of the endoscope arms 27 to 29 are each rotated around the rotation axis. The rotations of the optical adapters 274 to 294 on the distal end portions of the endoscope arms 27 to 29 are controlled by, for example, an endoscope arm drive controller 214. Specifically, for example, joints 273 to 293 each include a small (low speed) motor for rotating the distal end portion separately from a motor for driving the joint, and the distal end portions of the endoscope arms 27 to 29 can be rotated by a rotation drive mechanism (including a rotation drive shaft coupled to the distal end portion and various gears for transmitting rotation drive (for example, a gear such as a bevel gear couples the distal end portion to a shaft of the small motor in some cases)). For example, the endoscope arm drive controller 214 is configured to control a rotation speed and similar factor of the distal end portion. The rotation of each distal end portion of the endoscope arms 27 to 29 can be detected by a position detector (for example, an encoder). For example, respective position detectors disposed on joints closest to the respective distal end portions can be used.

The imaging controller 215 stores images obtained by the imaging apparatuses (imagers) 2741 to 2941 while rotating in an image memory (not illustrated) with time stamps.

An image processor 216 generates a composite image while performing matching among the images obtained by the imaging controller 215. A composite image generation process will be described later.

Similarly to the second embodiment, as illustrated in FIG. 6, the distal end portions of the endoscope arms 27 to 29 are configured to be covered with, for example, transparent sheaths 61 having rounded distal end portions. This prevents body tissue of the patient P from being damaged during surgery.

### <Composite Image Generation Process>

The composite image generation process in the third embodiment can be realized by combining the composite image generation process (FIG. 3) in the first embodiment with the composite image generation process (FIG. 8) in the second embodiment. For example, the composite image of the images obtained by taking while rotating each of the imaging apparatuses (imagers) 2741 to 2941 of the three or more endoscope arms 27 to 29 is generated in accordance with the process in FIG. 8. This generates three or more rotation composite images (also referred to as around view images). The three or more rotation composite images are further combined in accordance with the process in FIG. 3, thus generating a final composite image.

### <Low Resolution Mode and High Resolution Mode>

In this embodiment, similarly to the second embodiment, for example, the images obtained while rotating whilethe distal end portions of the endoscope arms 27 to 29 are provided in low resolution (low image quality: in other words, a first resolution or a first image quality), and therefore, the composite image of the rotation images of the respective distal end portions and the final composite image obtained by further joining the three or more composite images are also indicated on the display screen of the display apparatus 30 with low resolution. On the other hand, similarly to the second embodiment, the images obtained while stopping the rotation of the distal end portions are indicated on the display screen of the display apparatus 30 with high resolution (high image quality: in other words, a second resolution or a second image quality (higher resolution compared with the first resolution)).

For example, when the operator O finds an anxious part and the like on the final composite image and wants to intensively observe the part, the operator O can manipulate the console apparatus 10 to instruct to stop the rotation of at least one distal end portion covering the part to be intensively observed among the distal end portions of the three or more endoscope arms 27 to 29, and instruct the part desired to be further intensively observed to confirm the part on the high resolution image.

### (4) Modification

(i) While, in the first to the third embodiments, the composite image indicating a current state in the body (for example, in the abdominal cavity or in the thoracic cavity) of the patient during surgery is displayed on the display screen of the display apparatus 30, past images (irrespective of the composite image or not) may be displayed on the display screen together (for example, on a multi-screen). For example, a group of the images from a time point where the distal end portions (the portions on which the imaging apparatuses (imagers) are mounted) of the endoscope arms 22 to 24 and the distal end portions of the endoscope arms 27 to 29 are inserted into the body of the patient P to a time point where those distal end portions move to a position for taking images of the surgical site are stored in the image memory (not illustrated). Therefore, displaying the images near insertion routes of the respective endoscope arms and the images on peripheries of insertion routes of surgical instruments mounted on the respective surgical arms (both are images taken in the past) with the current images during surgery on the display screen ensures, for example, the operator O to confirm whether the surgical instruments manipulated by the operator O himself/herself are inserted excessively pressing the organs in the body of the patient P.

The functions and the configurations in each embodiment may be appropriately combined for use. For example, when the endoscope apparatus includes the three or more endoscope arms 22 to 24, it is not necessary to configure every distal end portion of the three or more endoscope arms to be rotatable (a configuration where the distal end portion of at least one endoscope arm is rotatable may be employed).

(ii) The functions of each embodiment can be realized by program codes of software. In this case, a storage medium in which the program codes are recorded is provided to a system or an apparatus, and a computer (or a CPU and/or an MPU) in the system or the apparatus reads the program codes stored in the storage medium. In this case, the program code itself read from the storage medium realizes the above-described function of the embodiment, and the program code itself and the storage medium that stores the program code are included in this embodiment. As the storage medium for supplying such program codes, for example, a flexible disk, a CD-ROM, a DVD-ROM, a hard disk, an optical disk, a magneto-optical disk, a CD-R, a magnetic tape, a non-volatile memory card, and a ROM are used.

Based on the instruction of the program code, an OS (operating system) operating on a computer or similar system performs a part of or all the actual process, and with the process, the above-described function of the embodiment may be achieved. Furthermore, the program code read from the storage medium is written on a memory on the computer, subsequently, based on the instruction of this program code, a CPU or the like of the computer performs a part of or all the actual process, and with the process, the above-described function of the embodiment may be achieved.

Furthermore, the program code of the software achieving the function of the embodiment is delivered via a network, the program code is stored in the storage means such as the hard disk and the memory or the storage medium such as the CD-RW and the CD-R in the system or the apparatus, and the computer (or the CPU and/or the MPU) in the system or the apparatus may read and execute the program code stored in the storage means or the storage medium when used.

### REFERENCE SIGNS LIST

1 Surgical system
10 Console apparatus
20 Patient-side cart
21 Endoscope apparatus
22, 23, 24, 27, 28, 29 Endoscope arm
25, 26 Surgical arm
30 Display apparatus

1. An endoscope apparatus comprising:
one or more endoscopes each of that includes an imager on a distal end portion of a tubular housing, and obtains an image in a body of a subject;
one or more endoscope arms each of that includes a plurality of joints and a plurality of position detectors corresponding to the respective plural joints, and holds the endoscope,
a controller that processes a plurality of images obtained by the one or more endoscopes,
wherein the controller obtains position information of the imager by using the plurality of position detectors, and generates a composite image by joining the respective images together by using the position information and feature points of the respective images obtained by the one or more imagers.

2. The endoscope apparatus according to claim 1, comprising:
three or more of the endoscopes,
wherein the controller is electrically coupled to the three or more endoscopes, and processes the images obtained by the respective three or more endoscopes, and
the plurality of images are obtained by the imagers of the three or more endoscopes.

3. The endoscope apparatus according to claim 2,
wherein at least three images obtained by the three or more endoscopes have mutually overlapping regions, and
the controller specifies the mutually overlapping regions by using the feature points of the respective images, and generates the composite image.

4. The endoscope apparatus according to claim 2,
wherein the imagers included in the three or more endoscopes each include a lens having a viewing angle equal to or more than 90 degrees.

5. The endoscope apparatus according to claim 2, further comprising,
a memory to store images,
wherein the each of imagers obtains the image by each of the endoscopes at predetermined time intervals,
the controller stores, in the memory, the respective images together with time information when the respective images has obtained,
reads out the respective images having common time information from the memory,
generates a composite image from the read out images, and
stores the composite image and the time information in the memory,
wherein the controller reads out the composite image having predetermined time information from the memory in response to an input instruction, and
displays the composite image on the display screen in response to the input instruction.

6. The endoscope apparatus according to claim 1, comprising,
a rotation driver configured to rotate at least the distal end portion of the tubular housing of the endo scope,
wherein the imager has an optical axis having a predetermined angle with a rotation axis of the distal end portion of the tubular housing, and
the plurality of images are obtained by the imager by rotating the distal end portion of the tubular housing of the endo scope.

7. The endoscope apparatus according to claim 6,
wherein the rotation axis extends in a direction identical to a longitudinal direction of the tubular housing, and
the imager is disposed on the distal end portion of the tubular housing, the imager is inclined with respect to the rotation axis, and the optical axis of the imager has the predetermined angle with the rotation axis.

8. The endoscope apparatus according to claim 6,
wherein the predetermined angle between the optical axis of the imager and the rotation axis is ½ of a viewing angle of a lens of the imager.

9. The endoscope apparatus according to claim 6,
wherein the lens of the imager is a foveal lens.

10. The endoscope apparatus according to claim 6,
wherein a sheath is mounted on the distal end portion of the tubular housing.

11. The endoscope apparatus according to claim 10,
wherein the sheath has a rounded distal end portion, and the sheath is transparent.

12. The endoscope apparatus according to claim 6, comprising
three or more of the endoscopes,
wherein the plurality of images are obtained by the respective imagers while rotating the distal end portions of the respective tubular housings of the three or more endoscopes.

13. The endoscope apparatus according to claim 6,
wherein the controller stops the rotation of the distal end portions of the respective tubular housings in response to an input instruction, and obtains the plurality of images by at least one of the imagers in a state where the rotation is stopped.

14. The endoscope apparatus according to claim 13,
wherein the image obtained and indicated in the state where the rotation is stopped has a high image quality compared with the images obtained while rotating the distal end portions of the respective tubular housings.

15. The endoscope apparatus according to claim 13,
wherein the input instruction includes information on position of a target in the body, an image of the target being obtained in the state where the rotation is stopped, and
the controller obtains an image obtained by only the imager with which the image of the target position is obtainable.

16. An endoscope system, comprising:
one or more endoscopes each of that includes an imager on a distal end portion of a tubular housing, and obtains an image in a body of a subject;
a robot cart that includes an endoscope apparatus, the endoscope apparatus including one or more endoscope arms each of that includes a plurality of joints and a plurality of position detectors corresponding to the respective plural joints, and holds the endoscope;
a console apparatus that transmits an instruction to manipulate the endoscope arm; and
a display apparatus that displays an image taken by the endoscope apparatus on a display screen,
wherein the endoscope apparatus includes a controller that processes the plurality of images obtained by the one or more of endoscopes,
wherein the controller obtains position information of the imager by using the position detectors, and generates a composite image by joining the respective images together by using the position information and feature points of the respective images obtained by the one or more imagers.

17. The endoscope system according to claim 16,
wherein the endoscope apparatus includes three or more of the endoscopes,
the plurality of images are obtained by the imagers of the three or more endoscopes.

18. The endoscope system according to claim 16,
wherein the endoscope apparatus includes a rotation driver configured to rotate at least the distal end portion of the tubular housing of the endoscope,
wherein the imager has an optical axis having a predetermined angle with a rotation axis of the distal end portion of the tubular housing, and
the plurality of images are obtained by the imager by rotating the distal end portion of the tubular housing of the endoscope.

19. The endoscope system according to claim 18,
wherein the endoscope apparatus includes three or more of the endoscopes,
the rotation driver rotates at least the respective distal end portions of the tubular housings of the three or more endoscopes,
the imagers of the three or more endoscopes each have an optical axis having a predetermined angle with a rotation axis of the distal end portion of the corresponding tubular housing, and
the plurality of images are obtained by the respective imagers while rotating the distal end portions of the respective tubular housings of the three or more endoscopes.

20. A surgical system comprising,
the endoscope system according to claim 16,
wherein the robot cart further includes a surgical arm on which a surgical instrument is mounted, and
the console apparatus transmits an instruction to manipulate the surgical arm.
